(19) United States
(12) Patent Application Publication
(10) Pub. No.: US 2023/0190342 A1
(43) Pub. Date: Jun. 22, 2023

(54) SURGICAL DEVICE

(71) Applicant: Mark Robert BRINKER, Houston, TX (US)

(72) Inventor: Mark Robert BRINKER, Houston, TX (US)

(21) Appl. No.: 17/555,275

(22) Filed: Dec. 17, 2021

(51) Int. Cl. A61B 17/74; A61B 17/84; A61B 17/80; A61B 17/70

(52) U.S. Cl. CPC A61B 17/744; A61B 17/84; A61B 17/8023; A61B 17/7002

(57) ABSTRACT

A modular system for a surgical device is provided. The modular system includes an adjustable-length portion having a first end and a second end, the first end spaced apart from the second end along a length of the adjustable-length portion, and the adjustable-length portion including an actuator configured to be activated to change the length of the adjustable-length portion. The adjustable-length portion is configured to be secured to one or more bones. The modular system further includes a first fixed-length portion configured to attach to the first end of the adjustable-length portion to form a first surgical device.

### BACKGROUND

### Field

Embodiments of the present disclosure generally relate to apparatuses and methods relating to the use of surgical devices.

### Description of the Related Art

An intramedullary nail is most commonly utilized to stabilize a patient's fractured bone to promote fracture healing and early patient mobilization. Although these nails are most commonly surgically implanted in the medullary canal of a long bone, they can be implanted in other anatomic locations, including, but not limited to the outer periosteal surface of bone. Nails designed to stabilize fractures come in a variety of shapes and dimensions and are generally monolithic in construction. Most intramedullary nails intended for fracture stabilization have provisions for placing interlocking screws above and below the fracture site. Interlocking screws discourage/prevent axial and rotational motion between fracture segments and therefore promote stability and fracture healing. For the purpose of this disclosure the terms intramedullary nail, intramedullary rod, nail, and rod will have the same meaning.

The size (e.g., length and diameter) and shape of a given bone (e.g., a femur) can vary substantially from patient to patient. Furthermore, bones are generally not straight, so that intramedullary nails are commonly manufactured with one or more curved portions to assist in obtaining proper placement of the nail in the patient's bone. Additionally, for a given bone, such as the femur, there may be three or more locations on the bone through which the intramedullary nail can be inserted. Moreover, there may also be particular needs for more than one interlocking screw configuration for each of these intramedullary nail locations. Thus, a large number of intramedullary nails would be needed if a medical provider wanted to keep every type and size of intramedullary nail in stock. Fortunately, the cost associated with intramedullary nails designed for fracture stabilization is such that medical service providers are able to keep all of the various intramedullary nail types and sizes in their inventory.

Unlike intramedullary nails designed for fracture stabilization, more advanced intramedullary nails have been designed to allow bone segments to move relative to one another for the purpose of bone lengthening, bone shortening, or to eliminate a bone defect. These intramedullary nails include a portion that can extend, retract, or slide to assist in moving bone segments relative to one another to shorten, lengthen, or eliminate a bone defect. For example, a lengthening intramedullary nail can be extended by, for example, 0.75 to 1.00 mm a day for a period of time (e.g., 1 month) to allow the bone to slowly increase to the desired length.

Another type of intramedullary nail is a bone transport nail. These bone transport nails have been developed to reconstruct bone defects without the need of external fixation and without necessarily altering the length of the affected bone.

Just like the situation with intramedullary nails designed to stabilize fractures, the inventory requirement for lengthening nails is substantial to account for the various types of nails, anatomic locations, entry sites, and nail dimensions. Unfortunately, the cost associated with lengthening nails is many fold higher than fracture fixation nails and it is not practical for medical service providers to keep all of these lengthening nails in their inventory. Instead, medical service providers generally order the lengthening nail(s) needed for a specific patient. Because the exact dimensions of the nail needed can sometimes only be determined at the time of surgical implantation, medical providers generally order a range of sizes and dimensions which may require 10 to 20 nails be made available. The requirement to order specific intramedullary nails needed for a given patient often results in delays in treatment and can often result in a compromised outcome for the patient.

Therefore, there is a need for improvement to address the problems mentioned above. Some additional background information is provided below.

Limb lengthening is a process which harnesses the body's own innate ability to regenerate, form, and grow new bone and soft tissues which include blood vessels, nerves, and others. Limb lengthening is indicated in patients with: 1) congenitally short limbs; 2) developmentally short limbs; 3) shortening following bone loss from trauma; 4) shortening following bone loss from infection; 5) shortening following bone loss from tumor; 6) short stature; and 7) other conditions.

The process of limb lengthening begins with an evaluation of the patient's clinical condition. Radiographs are measured to determine the extent of limb lengthening required. In the case of one short limb, the contralateral limb is measured and serves as a template for a target length for the short limb.

Bones which can be lengthened in an upper limb include the clavicle, humerus, radius, ulna, and bones of the wrist, hand, and fingers. Bones which can be lengthened in a lower limb include the femur, tibia, fibula and the bones of the ankle, foot, and toes. It is also possible to lengthen and shorten the ribs or the bones of the spine or to distract and compress the soft tissues surrounding the spine to cause a crooked or curved spine to straighten.

The process of limb lengthening involves a surgical procedure wherein the bone or bones to be lengthened are cut or divided using a technique known as an osteotomy. This technique involves using one or a combination of methods to completely divide the bone. The osteotomy can be performed with any of the following methods used alone or in combination with one or more of the other methods and include an osteotomy performed: 1) via use of a surgical saw; 2) via use of a sharp surgical chisel known as an osteotome; 3) via use of multiple drill holes used to weaken the bone to allow it to be easily broken/cracked; 4) via a sharp hand-powered cutting wire known as a Gigli saw; and 5) via various other methods.

Once the bone has been broken via osteotomy, the bone is stabilized using one of several types of devices that fit into the two broad categories of external fixation and internal fixation. For the purpose of this disclosure the terms fracture and osteotomy will sometimes be used interchangeably.

An external fixator is an externally applied device that is attached to any of the following bone fixation pins and wires alone or in combination and include: Schanz Screws, Steinmann Pins, smooth pins, threaded pins, half pins, and wires. These fixation pins traverse the soft tissues to become surgically affixed to each segment of the divided bone. Moving the components of the external fixator that are located outside the patient's body transfers force through the fixation pins and wires to cause the bone segments to move relative to one another. Disadvantages of external fixation include: 1) potential infection from fixation pins and wires that traverse from the bone, through the soft tissues, to the external environment surrounding the limb; 2) unsightly permanent scarring as the fixation pins and wires cut through the soft tissues as the bone segments move; 3) pain associated with fixation pins and wires cutting through the soft tissues as the bone segments move; and 4) that patients do not generally favor this type of treatment.

Because of the inherent disadvantages of using an external fixator, a variety of techniques using internal fixation (e.g., a lengthening intramedullary nail or a lengthening extramedullary nail) have been developed.

A lengthening intramedullary nail is an internally implanted device including at least two telescopic components. The nail is powered in some manner, such as but not limited to being powered by: an electric motor, one or more rotatable magnets, memory metals, energy from rotation of the limb, hydraulics, gravitational forces, heat, and cold among others). A remote-control device can allow for control of the telescopic nail, for example to elongate a defined distance. These nails are most commonly surgically implanted inside the marrow cavity or medullary canal of a bone, but extramedullary positioning on or near the surface of the bone is another technique that can be used.

In addition to lengthening, these nails can also be remotely controlled to operate in the reverse manner so that the telescopic nail can be caused to shorten a defined distance. Although these surgical nails are commonly known as lengthening nails, these nails also have great clinical utility when used for shortening and/or bone transport. In this disclosure, the term “remotely movable surgical nail” is used to refer to a nail that can be remotely activated (e.g., through use of a remote control) to provide lengthening, shortening, or bone transport of a segment of bone to reconstruct a bony defect without necessarily lengthening or shortening the bone or limb.

These remotely movable surgical nails are commonly used in conjunction with interlocking screws, which are surgically implanted at the time the remotely movable surgical nail is implanted. Interlocking screws are fastened into drill holes in the bone that align with one or more engineered holes in the nail. By fastening each bone segment, for example after an osteotomy, with each end of the remotely movable surgical nail, the interlocking screws can provide the following benefits including: 1) preventing each bone segment from rotating around the telescopic nail; 2) preventing the bone segments from rotating relative to one another; 3) and allowing force to be transmitted from the lengthening nail to the bone for effective lengthening, shortening, or bone transport using the remote control.

In addition to treating deformities involving pure axial length, adjunctive surgical techniques and specially designed nails have been described, which also allow for correction of associated deformities of angulation, rotation, and translation.

Using a surgical nail to controllably shorten a bone may be useful in the following clinical situations: 1) compression of a fracture nonunion (i.e., a fracture of bone that is refusing to heal); 2) achieving bone to bone contact and compression in an acute or subacute fracture including cases where there is bone loss; 3) achieving bone to bone contact and compression in an acute or subacute fracture where there is bony comminution (i.e., multiple bone fragments); 4) managing bone loss in cases of tumor or infection; 5) shortening a limb in a patient with a limb length discrepancy; 6) patients with segmental nerve injury where shortening a limb allows for direct nerve repair; and 7) patients with segmental vessel injury where shortening a limb allows for direct vascular repair; 8) other various clinical situations.

In certain clinical situations it is desirable to alternatively lengthen and shorten a bone with a lengthening nail to promote healing. This alternating shortening and lengthening is sometimes referred to as the accordion maneuver or accordion technique.

Following a bone lengthening surgical procedure there is a latency phase, wherein no lengthening is performed. Depending on the specific clinical circumstances and medical provider preferences, the latency phase is typically 5-10 days. Following the latency phase, the active phase commences. In the case of limb lengthening, this active phase is commonly known as the distraction phase. During distraction, bone (commonly known as bony regenerate) is formed in the daily widening gap as the bone is slowly and gradually lengthened. The process of bone formation in this gap is known as distraction osteogenesis. The mechanism is that tension stress at the osteotomy site causes the formation of bone which is often from intramembraneous bone formation. The typical rate of distraction is 0.75 mm to 1.0 mm per day and the typical rhythm is such that this total daily distraction is achieved in 3 or 4 sessions of distraction of 0.25 mm per session. As the gap widens the body continues to grow tissue, including bone and the surrounding soft tissues which are also experiencing tension stress.

Once the bone(s)/limb(s) has/have reached the desired length, active distraction ceases, and the consolidation phase begins. During the consolidation phase the bony regenerate matures and hardens. Ultimately this bone will be indistinguishable from the surrounding bone and will have similar mechanical strength characteristics.

The overall limb-lengthening process takes months and naturally the greater the lengthening required, the longer the process takes.

Another group of patients who are candidates for distraction osteogenesis are those with severe extremity problems who are sometimes even at risk for needing an amputation. These patients typically have significant bone loss related to trauma, infection, tumor, or congenital or developmental conditions, with or without associated soft tissue loss.

Whereas distraction and compression as described above involve lengthening or shortening at a single healing site, a technique known as bone transport has been developed to reconstruct bone defects without necessarily altering the length of the affected limb. Specifically, when utilizing the strategy of bone transport an osteotomy is performed at a site distant from the bone defect. Once the latency phase has ended, the bone segment adjacent to the osteotomy is transported typically at a rate of 0.75 mm to 1.0 mm per day towards the area of the defect. As transport continues, the original bone defect becomes smaller each day and the osteotomy gap becomes wider and fills with new bone due to the phenomenon of distraction osteogenesis. In the earliest days of bone transport, the movement of the transport segment was powered by an external fixator. More recently, specially designed telescopic bone transport nails have been introduced and allow for bone transport without the requirement of external fixation.

Bone transport nails have been developed to reconstruct bone defects without necessarily altering the length of the affected bone. Specifically, when utilizing the strategy of bone transport an osteotomy is performed at a site distant from the bone defect. The bone transport nail is constructed with an outer cylindrical portion and an inner movable portion. The bone transport nail is typically implanted in the medullary canal of a bone, although extramedullary locations are also possible. Once the transport nail has been implanted, interlocking screws are positioned at the ends of the outer nail to hold the bone at a fixed length. Following an osteotomy at a distant site from the bone defect or fracture site, one or more additional interlocking screws are positioned through engineered holes in the inner portion to fixate the transport segment of bone. As the inner portion moves at the command of a remote-control device, force is transmitted to the bone through the interlocking screws and the transport segment of bone is caused to move. As transport continues and the transport segment moves progressively away from the osteotomy site, the original bone defect becomes smaller each day and the osteotomy gap becomes wider and fills with new bone due to the phenomenon of distraction osteogenesis. Eventually the transport segment docks, the bone defect having been substantially eliminated, and bone to bone healing ensues. It is not uncommon for practitioners to bone graft the docking site.

As previously mentioned, remotely movable surgical nails can also be implanted in locations outside the marrow cavity in an extramedullary anatomic location. One such application involves implantation of one or more remotely movable extramedullary nails or rods in and around the vertebral column to treat scoliosis (curvature of the spine). Using these remotely movable extramedullary nails/rods and applying distraction and/or compression at different rates and to varying degrees, a crooked vertebral column and spine can be gradually straightened. Just like the situation with intramedullary nails designed to stabilize fractures as well as lengthening, shortening, and bone transport nails, the inventory requirement for spinal lengthening nails/rods is substantial to account for the various types of nails, anatomic locations, and nail dimensions. Unfortunately, the cost associated with remotely movable spinal nails is many fold higher than fracture fixation nails and it is not practical for medical service providers to keep all of these nails in their inventory and these implants therefore are associated with the same availability problems of the remotely movable intramedullary nails used to treat patient's extremities.

The current state of the art for limb lengthening is the use of internal telescopic lengthening nails (e.g., intramedullary nails). As described above, these nails and other similar remotely movable surgical nails allow for controllable limb lengthening, limb shortening, and bone transport in cases of major bone defect. Recently, remotely movable plates have also been introduced that allow for controllable limb lengthening, limb shortening, and bone transport in cases of major bone defect. These plates are typically implanted on and affixed to the outer surface of a long bone, although intramedullary implantation is also possible.

Because of their telescopic purpose and function, remotely movable surgical nails (e.g., lengthening nails) are often designed with a cylinder within a cylinder construction. This is in distinct contrast to medullary nails which are designed to stabilize fractures and are manufactured with an anatomic bow or radius of curvature that matches the specific bow of the bone it is intended to stabilize. Remotely movable surgical nails manufactured with an anatomic bow or curvature are also possible.

When inserting a remotely movable surgical nail (e.g., a lengthening nail) into a bone, great consideration is needed to use the appropriate sized nail. Size considerations include nail length and diameter. A nail too large can lead to injury or fracture of the bone during nail insertion. Specifically, an oversized lengthening nail can penetrate and fracture a bone as the nail is advanced down the medullary canal. Conversely, a nail too small can lead to instability at the osteotomy site which can be associated with pain and poor formation of bony regenerate.

It must be understood that standard fracture nails, lengthening/shortening nails, and bone transport nails are used in several long bones of the upper and lower extremity. These bones include but are not limited to the following: 1) clavicle; 2) humerus; 3) radius; 4) ulna; 5) metacarpals; 6) femur; 7) tibia; 8) fibula; 9) metatarsals; and 10) others. It should also be understood that spinal lengthening nails can be used in the following regions of the spine: 1) cervical; 2) thoracic; 3) lumbar; 4) sacrum; as well as the ribs.

It should further be understood that the medullary/marrow canal/cavity of several of the aforementioned long bones can be entered from a variety of starting points. As just one example, the medullary canal of the femur can be entered at the top of the femur (the proximal end) through either the tip of the greater trochanter or through the piriformis fossa. The medullary canal of the femur can also be entered at the bottom of the femur (the distal end) through the intercondylar notch. Some of these so-called entry or starting points require a non-cylindrical portion of the nail with a specific bend or architecture that allows the nail to pass into the bone without causing fracture of the bone at the entry site as well as preventing injury to neighboring anatomic structures. A specific bend is often (but not always) positioned on the portion of the nail that is the last portion to enter the bone upon insertion into the medullary canal.

In addition, remotely movable surgical nails (e.g., lengthening nails) for each bone must allow for a variety of possible locking screw configurations. This often means that the same nail must be manufactured in duplicates excepting that the locking screw configuration is different.

All of the above necessarily means that huge inventory is required to meet the wide variety of clinical circumstances and demands of different remotely lengthening/shortening nails, bone transport nails, and spinal lengthening nails/rods. As just one example, one manufacturer of lengthening nails for the long bones of the upper and lower extremities offers a total of six different nail architectures for various bones and starting points. When accounting for the various bones, starting points, nail lengths and diameters needed to treat a wide array of clinical conditions and patient dimensions, this manufacturer currently produces a total of 97 unique lengthening nails. In addition, this same manufacturer produces a total of 105 bone transport nails. As mentioned above, due to the high cost of these surgical nails, medical service providers typically do not keep all of these intramedullary nails in their inventory, and instead generally order the specific surgical nail needed for a specific patient. The requirement to order the specific lengthening nail needed for a given patient often results in delays and can often extend the time period in which a patient is suffering from their condition. Therefore, there is a need for improvement to address the problems mentioned above.

### SUMMARY OF THE INVENTION

Embodiments of the present disclosure generally relate to apparatuses and methods relating to the use of surgical devices that allow for bone lengthening, bone shortening, and bone transport.

In one embodiment, a modular system for a surgical device is provided. The modular system includes an adjustable-length portion having a first end and a second end, the first end spaced apart from the second end along a length of the adjustable-length portion, and the adjustable-length portion including an actuator configured to be activated to change the length of the adjustable-length portion. The adjustable-length portion is configured to be secured to one or more bones. The modular system further includes a first fixed-length portion configured to attach to the first end of the adjustable-length portion to form a first surgical device.

In another embodiment, a modular system for a surgical device is provided. The modular system includes an adjustable-length portion having a first end and a second end, the first end spaced apart from the second end along a length of the adjustable-length portion, the adjustable-length portion configured to be secured to one or more bones, and the adjustable-length portion including an actuator configured to be activated to change the length of the adjustable-length portion by moving the second end; and a first fixed-length portion configured to attach to the second end of the adjustable-length portion to form a first surgical device.

In another embodiment, a modular system for a surgical device is provided. The modular system includes a first portion; and a second portion having a first end and a second end, the second portion including a first part, a second part, and an actuator configured to move the first part relative to the second part, wherein the second portion is configured to be secured to one or more bones, and the first portion is configured to attach to the first end of the second portion to form a first surgical device.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1A shows an intramedullary nail positioned inside a femur, according to one embodiment.

FIG. 1B shows the intramedullary nail of FIG. 1A with the fixed-length portion separated from the adjustable-length portion, according to one embodiment.

FIG. 1C is a close-up of section 1C in FIG. 1B and shows additional detail of the intramedullary nail, according to one embodiment.

FIG. 1D shows an alternative intramedullary nail, according to one embodiment.

FIG. 2 illustrates an intramedullary nail positioned inside the femur, according to another embodiment.

FIG. 3A illustrates an intramedullary nail positioned inside the femur, according to another embodiment.

FIG. 3B illustrates an intramedullary nail positioned inside the femur, according to another embodiment.

FIG. 4 illustrates an intramedullary nail positioned inside a femur, according to another embodiment.

FIG. 5 illustrates an intramedullary nail positioned inside a tibia, according to another embodiment.

FIG. 6 illustrates an intramedullary nail positioned inside a humerus, according to another embodiment.

FIG. 7 illustrates an intramedullary nail positioned inside the femur, according to another embodiment.

FIG. 8 illustrates an intramedullary nail positioned inside the femur, according to another embodiment.

FIG. 9 illustrates a pair of spinal rods, according to another embodiment.

FIG. 10 illustrates a surgical plate, according to another embodiment.

To facilitate understanding, identical reference numerals have been used, where possible, to designate identical elements that are common to the figures. It is contemplated that elements disclosed in one embodiment may be beneficially utilized on other embodiments without specific recitation. The drawings referred to here should not be understood as being drawn to scale unless specifically noted. Also, the drawings are often simplified, and details or components omitted for clarity of presentation and explanation. The drawings and discussion serve to explain principles discussed below, where like designations denote like elements.

### DETAILED DESCRIPTION

Embodiments of the present disclosure generally relate to apparatuses and methods relating to the use of surgical nails.

Although the bulk of the following description of the Figures generally refers to use of different intramedullary nails in a human femur, the advantages of this disclosure can be applied to any bone in which an intramedullary nail can be used. Non-limiting examples of other bones for which the advantages of this disclosure can be applied can include the clavicle, humerus, radius, ulna, bones of the wrist, hand, and fingers as well as the tibia, fibula and the bones of the ankle, foot, and toes.

Additionally, the advantages of the disclosure can apply to other surgical devices, such as extramedullary rods (e.g., spinal rods) and surgical plates that, for example, can be positioned on the outer periosteal surface of bones for stabilizing and/or moving bone segments. Additionally, the use of the term “nail” is not meant to be limiting and the advantages of this disclosure can be applied to any surgical device, such as a rod or a plate, which can be used to mechanically move or stabilize one or more segments of a bone relative to another segment of that bone or another bone. The following embodiments describe intramedullary and extramedullary surgical devices that can move one or more sections of one or more bones by performing procedures, such as bone lengthening, bone shortening, or bone transport. The following embodiments can also be used to stabilize one or more sections or segments of one or more bones.

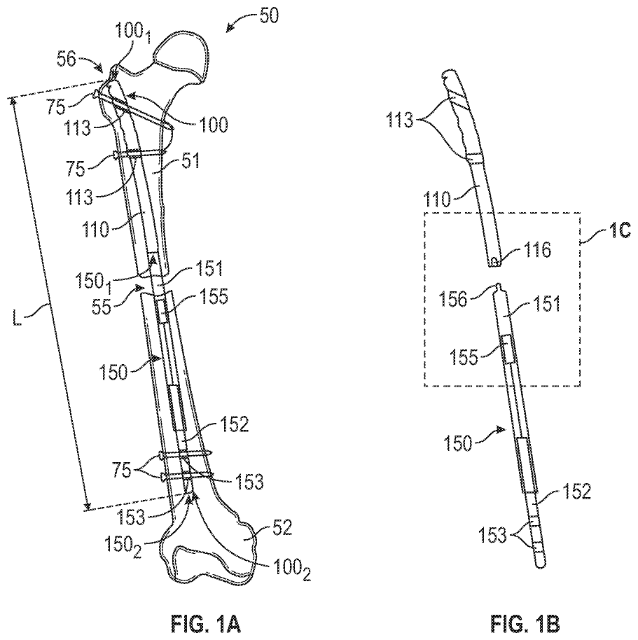

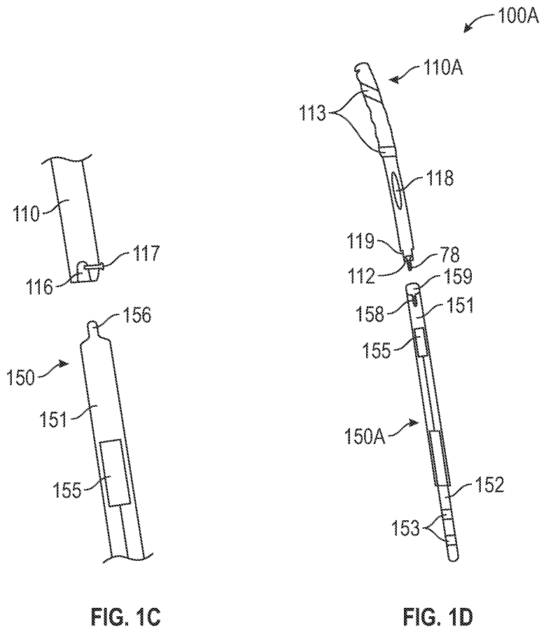

FIG. 1A shows an intramedullary nail 100 positioned inside a femur 50, according to one embodiment. The intramedullary nail 100 can be referred to as a trochanteric-entry antegrade femoral nail as the intramedullary nail 100 has been inserted into the femur 50 through the greater trochanter 56 of the femur 50. The femur 50 is broken along a fracture 55 that separates the femur 50 into an upper section 51 and a lower section 52. The intramedullary nail 100 can be used to move the lower section 52 relative to the upper section 51 for a variety of reasons, such as to lengthen, shorten, or assist in removing a bone defect that could be present at the fracture 55 or another location.

The intramedullary nail 100 includes a fixed-length portion 110 and an adjustable-length portion 150. The intramedullary nail 100 has a length L extending from a first end 1001 on the fixed-length portion 110 to an opposing second end 1002 on the adjustable-length portion 150. The length L is the largest dimension of the intramedullary nail 100. When the lengths of other surgical devices are described below, these lengths also refer to the largest dimension of those surgical devices. Each portion 110, 150 is secured to the femur 50 with fasteners 75 (e.g., interlocking screws). The fixed-length portion 110 includes holes 113. The fasteners 75 extend through the holes 113 to secure the fixed-length portion 110 to the upper section 51 of the femur 50.

Used herein, the term fixed-length (e.g., fixed-length portion 110) refers to a length of a component that cannot be remotely changed (e.g., through use of a remote control) after the component has been inserted into the patient. The term fixed-length does not refer to a component that has a length that cannot be changed before the component is inserted into a patient. For example, all of the fixed-length portions described herein (e.g., fixed-length portion 110) can include adjustable parts (e.g., telescopic components) that could be mechanically adjusted by a user before insertion of the fixed-length portion into a patient. For example, a single fixed-length portion (e.g., fixed-length portion 110) could have a length that is adjustable by sliding inner and outer telescopic components relative to each other before insertion of the portion into the patient. The adjustment of such fixed-length portions is another way that inventory costs can be reduced.

The adjustable-length portion 150 includes a first end 1501 and a second end 1502. The first end 1501 is spaced apart from the second end 1502 along a length of the adjustable-length portion 150. This length that spaces apart the first end 1501 from the second end 1502 corresponds to or substantially corresponds to the same direction in which the length of the adjustable-length portion 150 can be adjusted.

The adjustable-length portion 150 includes a base portion 151 and an extendable portion 152. The extendable portion 152 can move relative to the base portion 151, for example using a telescopic arrangement. For example, the extendable portion 152 can extend or retract relative to the base portion 151. The extendable portion 152 includes holes 153. The fasteners 75 extend through the holes 153 to secure the extendable portion 152 to the lower section 52 of the femur 50.

The base portion 151 includes an actuator 155. The actuator 155 can be activated to move the extendable portion 152 relative to the base portion 151. After the intramedullary nail 100 is secured to the femur 50 as shown, the movement of the extendable portion 152 initiated by the actuator 155 can be used to achieve the desired outcome (e.g., lengthening, shortening, or defect removal). The actuator 155 can be any type of actuator suitable for creating movement (e.g., extension or retraction) of the extendable portion 152 relative to the base portion 151. Examples of suitable actuators that can be used as the actuator 155 include, but are not limited to, a magnetic actuator, hydraulic actuator, heat or cold sensitive actuator, pressure sensitive actuator, motion sensitive actuator, or an electric motor. Many of the actuators that can be used as the actuator 155 can be remotely activated from outside of the patient's body.

The fixed-length portion 110 is removably attached to the adjustable-length portion 150. FIG. 1B shows the intramedullary nail 100 with the fixed-length portion 110 separated from the adjustable-length portion 150, according to one embodiment. FIG. 1C is a close-up of section 1C in FIG. 1B and shows additional detail of the intramedullary nail 100, according to one embodiment. As shown in FIG. 1B, the adjustable-length portion 150 includes an extension 156 that fits into a corresponding recess 116 on the fixed-length portion 110. In some embodiments, the recess 116 can be a slot, hole, or any other arrangement configured to receive the extension 156. Referring to FIG. 1C, the adjustable-length portion 150 can be secured to the fixed-length portion 110 through use of a set screw 117 extending through a side of the fixed-length portion 110, so that the set screw 117 can hold the extension 156 in the recess 116. In some embodiments, the extension 156 can include a recess (not shown) to provide a more secure connection for the set screw 117. In other embodiments, the fixed-length portion 110 can be removably attached to the adjustable-length portion 150 through other means, such as through the use of corresponding male and female threaded connections on the different portions 110, 150.

FIG. 1D shows an alternative intramedullary nail 100A, according to one embodiment. The alternative intramedullary nail 100A is the same as the intramedullary nail 100 described above except that the alternative intramedullary nail 100A includes a different fixed-length portion 110A and a different adjustable-length portion 150A. The differences between the fixed-length portion 110A and the adjustable-length portion 150A relative to the corresponding portions 110, 150 in the intramedullary nail 100 (see FIG. 1B) relate to the components used to connect the fixed-length portion 110A with the adjustable-length portion 150A.

For the alternative intramedullary nail 100A, a fastener 78 (e.g., a threaded fastener such as a screw) is inserted through the interior of the fixed-length portion 110A and connected to a receiving portion 158 (e.g., a threaded connection having internal threads) in the adjustable-length portion 150A. The fixed-length portion 110A can include a narrowed portion 119 with a hole 112 through which the fastener 78 can extend through for making the connection with the adjustable-length portion 150A. The adjustable-length portion 150A can include a sleeve 159 for receiving the narrowed portion 119.

The fixed-length portion 110A can include an additional hole 118 or a slot (not shown) in a side surface of the fixed-length portion 110A. The hole 118 is configured to enable a tool (e.g., a screwdriver or driver) to be inserted into the interior of the fixed-length portion 110A, so that the fastener 78 can be used to connect the fixed-length portion 110A with the adjustable-length portion 150A. Hole 118 may be particularly useful in cases where the fixed-length portion 110A includes a bend. The fixed-length portion 110A is configured to be inserted into the greater trochanter 56 of the femur 50 (see FIG. 1A) and thus includes a bend. In embodiments in which the fixed-length portion 110A includes a bend, an alternative method of accessing the fastener 78 for the purpose of connecting the fixed-length portion 110A to the adjustable-length portion 150A is the use of a flexible tool (e.g., a flexible-shaft screwdriver or flexible-shaft driver) that can be inserted through the entire length of the fixed-length portion. For fixed-length portions not including a bend, a hole can alternatively be placed at the end of the fixed-length portion that does not connect with the adjustable-length portion. Due to the shape of a straight fixed-length portion, a tool (e.g., a screwdriver) can be easily inserted through the entire interior length of the fixed-length portion to enable the fixed-length portion to be connected to the adjustable-length portion, for example by using the fastener 78 and the receiving portion 158 shown in FIG. 1D.

The intramedullary nail 100A provides one example of an alternative method of connecting two portions of a surgical device, but this disclosure is not to be limited to the methods of connecting the fixed-length portion 110 to the adjustable-length portion 150 in FIGS. 1A-1C or for connecting the fixed-length portion 110A to the adjustable-length portion 150 in FIG. 1D. For all of the surgical devices described in this disclosure that include two or more parts to be connected, any method can be used to connect the two separate parts, such as fasteners, interlocking parts, press-fit connections, or connections that use adhesives, magnets, any other connections known.

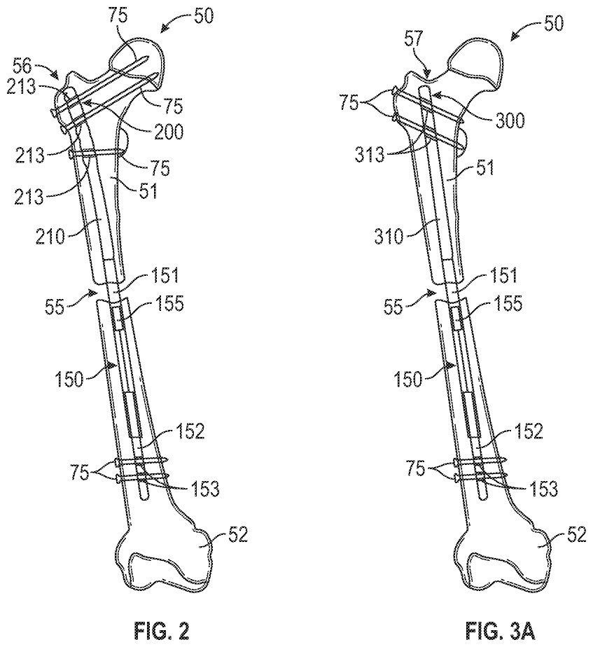

FIG. 2 illustrates an intramedullary nail 200 positioned inside the femur 50, according to another embodiment. The intramedullary nail 200 is the same as the intramedullary nail 100 described above except that the intramedullary nail 200 includes a fixed-length portion 210 in place of the fixed-length portion 110 in the intramedullary nail 100. The fixed-length portion 210 is the same as the fixed-length portion 110 except that the fixed-length portion 210 includes a different configuration of holes 213 for the fasteners 75 relative to the configuration of holes 113 used in the first portion 110 described above.

Although the same femur 50 is shown in FIG. 1A and FIG. 2 for ease of illustration, it is to be understood that there is at least some significant difference (e.g., site of bone fracture, injury pattern, bone shape, bone density, bone length, bone diameter, height of patient, weight of patient, age of patient, etc.) between the femur/patient from FIG. 1A relative to the femur/patient from FIG. 2 that would warrant the use of a different fastener configuration in the intramedullary nail 200 relative to the intramedullary nail 100.

Lengthening/shortening intramedullary nails, such as the intramedullary nails 100, 200 shown in FIGS. 1 and 2 can be quite expensive. Therefore, it is generally cost prohibitive for a medical service provider (e.g., a hospital in which orthopedic surgeries are performed, such as bone lengthening procedures) to keep the vast array of different types and sizes of intramedullary nails for each bone and patient in stock, such as keeping each of the intramedullary nails 100, 200 in stock. The bulk of the cost for the intramedullary nails is due to the cost of the components that can move the patient's bone. In this disclosure, the intramedullary nails 100, 200 have each included a respective fixed-length portion 110, 210 that does not include any movable parts. These fixed-length portions 110, 210 are relatively inexpensive when compared to the adjustable-length portion 150. Because each intramedullary nail 100, 200 includes the same adjustable-length portion 150 (i.e., the expensive portion) and because the fixed-length portions 110, 210 are relatively inexpensive, the medical service provider can keep both fixed-length portions 110, 210 and the adjustable-length portion 150 in stock. Furthermore, because each fixed-length portion 110, 210 can be easily attached to the adjustable-length portion 150, the medical service provider can evaluate their patient and quickly select and attach the correct fixed-length portion 110, 210 to use on the patient's procedure, such as a femur-lengthening procedure. Thus, having both fixed-length portions 110, 210 and the adjustable-length portion 150 in stock can reduce the likelihood of a patient having their procedure delayed due to waiting for the intramedullary nail to arrive from the manufacturer.

Thus, this disclosure provides a modular system allowing the medical service provider to provide two different types of surgery by keeping three different components in inventory. In FIGS. 1A and 2, the modular system includes the fixed-length portions 110, 210 and the one adjustable-length portion 150. As described below, the modular system can be expanded significantly allowing a large number of surgeries to be completed while keeping inventory costs down.

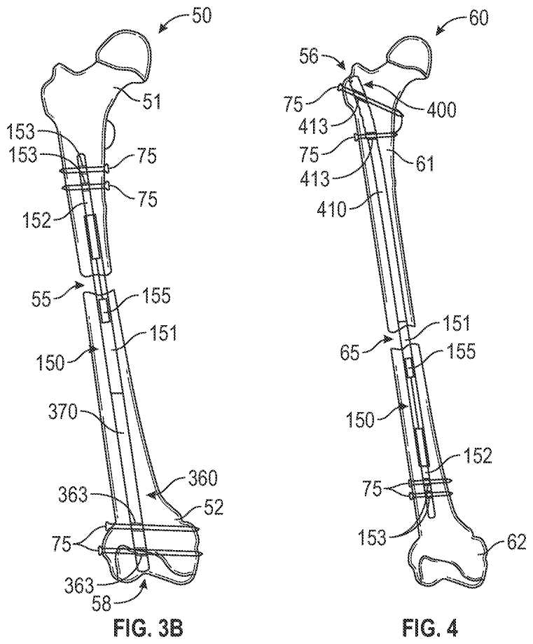

FIG. 3A illustrates an intramedullary nail 300 positioned inside the femur 50, according to another embodiment. The intramedullary nail 300 is the same as the intramedullary nail 100 (see FIG. 1A) described above except that the intramedullary nail 300 includes a fixed-length portion 310 in place of the fixed-length portion 110 that was part of the intramedullary nail 100. The fixed-length portion 310 can be quickly attached to the adjustable-length portion 150 in a similar manner as described above for the intramedullary nail 100 shown in FIG. 1A.

The fixed-length portion 310 has a different shape than the fixed-length portion 110. This shape difference is due to the anatomic area where the intramedullary nail 300 is inserted into the femur 50. The intramedullary nail 300 can be referred to as a piriformis-entry antegrade femoral nail as the intramedullary nail 300 has been inserted into the femur 50 through a portion 57 on top of the femur 50 in the region where the piriformis muscle attaches (the piriformis fossa) to the femur 50. The fixed-length portion 310 also includes a different set of holes 313 through which the fasteners 75 can extend to secure the fixed-length portion 310 to the upper section 51 of the femur 50.

Although the same femur 50 is shown in FIG. 3A and FIG. 1A for ease of illustration, it is to be understood that there is at least some significant difference (e.g., site of bone fracture, injury pattern, bone shape, bone density, bone length, bone diameter, height of patient, weight of patient, age of patient, etc.) between the femur/patient from FIG. 1A relative to the femur/patient from FIG. 3A that would warrant the use of a different fixed-length portion 310 in the intramedullary nail 300 when compared to the use of the fixed-length portion 110 used in the intramedullary nail 100.

Because the fixed-length portion 310 includes no movable parts, the fixed-length portion 310 can be relatively inexpensive like the fixed-length portions 110, 210 described above. Thus, the fixed-length portion 310 can be added to a medical service provider's inventory without significantly adding to the cost of the provider's inventory. When a medical service provider has each of the fixed-length portions 110, 210, 310 in stock, the medical service provider has the parts to quickly perform three different types of surgeries without waiting for parts from the manufacturer. Furthermore, because the fixed-length portions cost substantially less (e.g., a cost of a fixed-length portion can be only ten percent of the cost of an adjustable-length portion) than the adjustable portion 150, the medical service provider can keep the parts in stock to perform these three different types of surgery for substantially less cost than it would conventionally cost to only keep three intramedullary nails in stock. Thus, having the fixed-length portion 310 in inventory in addition to the fixed-length portions 110, 210 further reduces the likelihood of a patient having their procedure delayed due to waiting for the intramedullary nail to arrive from the manufacturer.

FIG. 3B illustrates an intramedullary nail 360 positioned inside the femur 50, according to another embodiment. The intramedullary nail 360 is the same as the intramedullary nail 100 (see FIG. 1A) described above except that the intramedullary nail 360 includes a fixed-length portion 370 in place of the fixed-length portion 110 that was part of the intramedullary nail 100. The fixed-length portion 370 can be quickly attached to the adjustable-length portion 150 in a similar manner as described above for the intramedullary nail 100 shown in FIG. 1A.

The fixed-length portion 370 has a different shape than the fixed-length portion 110. This shape difference is due to the anatomic area where the intramedullary nail 360 is inserted into the femur 50. The intramedullary nail 360 can be referred to as a retrograde femoral nail as the intramedullary nail 360 has been inserted into the femur 50 through a bottom portion 58 of the femur 50. Thus, the adjustable-length portion 150 can work in the opposite direction (e.g., extending upward) in the intramedullary nail 360 relative to the intramedullary nail 100 when both are used for a bone-lengthening procedure.

The fixed-length portion 370 also includes a different set of holes 363 relative to the holes 113 of the fixed-length portion 110 of the intramedullary nail 100. The fasteners 75 can extend through the holes 363 to secure the fixed-length portion 370 to the femur 50. The fixed-length portion 370 is secured to the lower section 52 of the femur 50. The adjustable-length portion 150 is secured to the upper section 51 of the femur 50.

Although the same femur 50 is shown in FIG. 3B and FIG. 1A for ease of illustration, it is to be understood that there is at least some significant difference (e.g., site of bone fracture, injury pattern, bone shape, bone density, bone length, bone diameter, height of patient, weight of patient, age of patient, etc.) between the femur/patient from FIG. 1A relative to the femur/patient from FIG. 3B that would warrant the use of a different fixed-length portion 370 in the intramedullary nail 360 when compared to the use of the fixed-length portion 110 used in the intramedullary nail 100.

In some embodiments, the fixed-length portion 370 may be the same as the fixed-length portion 310 (see FIG. 3A) except that the fixed-length portion 370 can include a different set of holes 363 for fasteners 75 compared to the set of holes 313 used for the fasteners 75 in the fixed-length portion 310. The intramedullary nail 360 is another example of how another fixed-length portion (i.e., fixed-length portion 370) can be added to the inventory to expand the modular system and allow for another surgery to performed with the same adjustable-length portion (i.e., the adjustable-length portion 150).

As shown in FIG. 1A, the intramedullary nail 100 is configured to at least be substantially fully inserted into the femur 50 (also referred to as first bone) through the greater trochanter 56 (first location) on the femur 50. Used herein, substantially fully inserted means that at least 90% of the length of the intramedullary nail (e.g., length L of intramedullary nail 100 in FIG. 1A) can be inserted into the patient's bone at a given location without causing harm other than any harm typically associated with proper insertion of an intramedullary nail in a patient. As shown in FIG. 3A, the intramedullary nail 300 is configured to at least be substantially fully inserted into the femur 50 through the portion 57 (second location) on top of the femur 50 in the region where the piriformis muscle attaches (the piriform is fossa) to the femur 50. On the other hand, the intramedullary nail 100 (FIG. 1A) is not configured to be substantially fully inserted into the femur 50 at the portion 57. Similarly, the intramedullary nail 300 (FIG. 3A) is not configured to be substantially fully inserted into the femur 50 at the greater trochanter 56.

In a similar fashion, the intramedullary nail 100 (see FIG. 1A) is not configured to be substantially fully inserted into the femur 50 through the bottom portion 58 (see FIG. 3B) of the femur 50. The intramedullary nail 100 is not configured to be substantially fully inserted into the femur 50 through the bottom portion 58 of the femur 50 due at least in part to the trajectory of the holes 113 of the intramedullary nail 100, which would place the fasteners 75 in a position that is anatomically improper and cause harm or injury to the patient. Similarly, the intramedullary nail 360 (FIG. 3B) is not configured to be substantially fully inserted into the femur 50 at the greater trochanter 56.

FIG. 4 illustrates an intramedullary nail 400 positioned inside a femur 60, according to another embodiment. The intramedullary nail 400 is the same as the intramedullary nail 100 (see FIG. 1A) described above except that the intramedullary nail 400 includes a fixed-length portion 410 in place of the fixed-length portion 110 that was part of the intramedullary nail 100. The fixed-length portion 410 of the intramedullary nail 400 is longer than the fixed-length portion 110 of the intramedullary nail 100.

The femur 60 in FIG. 4 is longer than the femur 50 described above (see e.g., FIG. 1A). The femur 60 is broken along a fracture 65 that separates the femur 60 into an upper section 61 and a lower section 62. The intramedullary nail 400 can be used to move the lower section 62 relative to the upper section 61 for a variety of reasons, such as to lengthen, shorten, or assist in removing a bone defect that could be present at the fracture 65 or another location. To accommodate this size difference between the femur 50 (FIG. 1A) and the longer femur 60 (FIG. 4), the intramedullary nail 400 includes the longer fixed-length portion 410 mentioned above. The fixed-length portion 410 can have holes 413 that may be different (e.g., larger to accommodate larger fasteners) than the holes 113 described above.

Thus, in order to be ready to perform the same type of surgery on two bones of different lengths, the medical service provider only needs to keep one expensive part (e.g., one adjustable-length portion 150) in stock. Obviously, there are more than two lengths of femurs, but in order for the medical service provider to be prepared for other lengths of femurs besides the femurs 50, 60 described herein, the provider only needs to obtain some additional inexpensive fixed-length portions for each length. Furthermore, the medical service provider can obtain a variety of fixed-length portions having different lengths for the other variations described above. For example, the provider can inexpensively obtain a variety of fixed-length portions having different lengths with the screw configuration described above in reference to FIG. 2. Similarly, the provider can inexpensively obtain a variety of fixed-length portions for the piriform is insertion described above in reference to FIG. 3A or for the retrograde insertion described above in reference to FIG. 3B.

Thus, it is easy to see how a medical service provider can be prepared to perform more than ten different types of surgeries while only keeping one expensive component (i.e., the adjustable-length portion 150) in stock. For example, if the intramedullary nails described in reference to FIGS. 1A, 2, 3A, and 3B can each be modified for four different lengths by using a fixed-length portion having a different length, then the medical service provider can inexpensively, be prepared to perform 16 different surgeries. That said, the number is actually significantly higher than 16 because the intramedullary nails having the piriformis insertion point or the retrograde insertion point could also have different screw configurations.

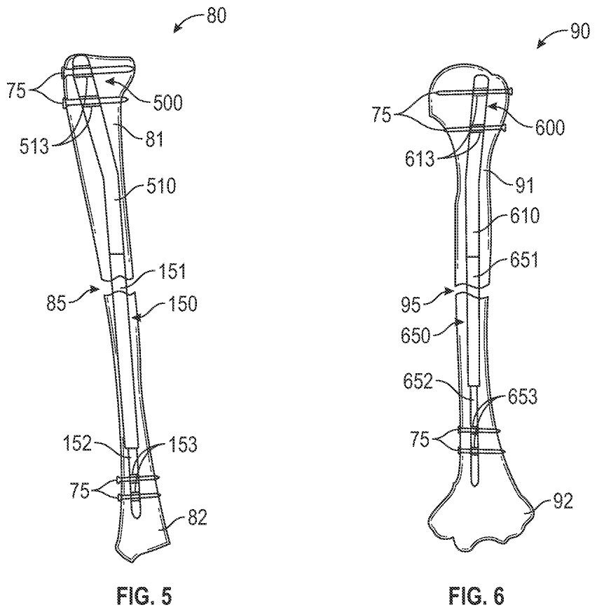

FIG. 5 illustrates an intramedullary nail 500 positioned inside a tibia 80, according to another embodiment. The intramedullary nail 500 is the same as the intramedullary nail 100 described above except that the intramedullary nail 500 includes a fixed-length portion 510 in place of the fixed-length portion 110 that was part of the intramedullary nail 100 (see FIG. 1A). The fixed-length portion 510 can include a set of holes 513 for fastening the fixed-length portion 510 to the tibia 80. The fixed-length portion 510 of intramedullary nail 500 has a particular bend (commonly known as a Herzog bend) which allows the intramedullary nail 500 to be inserted into the tibia 80 without causing damage or fracture to the tibia 80. As just one example, the intramedullary nail 100 of FIG. 1A is not configured to be inserted into the tibia 80 and would cause injury to the patient if this insertion was attempted. Despite being used for a different bone, the adjustable-length portion 150 in the intramedullary nail 500 for the tibia 80 is the same as the adjustable-length portion 150 in the intramedullary nail 100 (see FIG. 1A) used for the femur 50.

The tibia 80 is broken along a fracture 85 that separates the tibia 80 into an upper section 81 and a lower section 82. The intramedullary nail 500 can be used similarly to the nail 100 described above to move the lower section 82 relative to the upper section 81 for a variety of reasons, such as to lengthen, shorten, or assist in removing a bone defect that could be present at the fracture 85 or another location. The intramedullary nail 500 includes the adjustable-length portion 150 that includes the actuator 155 (see FIG. 1A) described above that is used to move the extendable portion 152 relative to the base portion 151. The actuator 155 and the connection of the actuator 155 to the extendable portion 152 are omitted in FIG. 5 for ease of illustration.

Femurs and tibias can often have a size and shape similar enough that similarly sized telescopic lengthening intramedullary nails can be used. Thus, with reference to FIG. 1A and FIG. 5 the same adjustable-length portion 150 can be used for the femur 50 and the tibia 80. Like intramedullary nails for the femur, intramedullary nails for the tibia can also have different screw configurations, different insertion points, and different lengths. Thus, it is easy to understand how a modular system including only one expensive component (i.e., the adjustable-length portion 150) that can be used on different bones (e.g., a femur and a tibia) can allow a medical service provider to perform a very large number of different types of surgeries while maintaining low inventory costs.

While the description above has described only one adjustable-length portion 150 to be used on the femur and tibia, adjustable-length portions having different sizes, (e.g., different diameters) different strokes (i.e., how far the extendable portion can extend or retract), and different shapes and curvatures are also envisioned. The benefits of this disclosure can be extended, so that a modular system can be generated for each of these different adjustable-length portions. Furthermore, in some instances, the modular system can be expanded further when the fixed-length portions described above can be attached to different adjustable-length portions. For example, some manufacturers often supply a set of three different intramedullary nails that can extend with a stroke of either 30 mm, 50 mm, or 80 mm. Thus, using this example, each fixed-length portion described above can be attached to three different adjustable-length portions. Extending the modular system to allow for the fixed-length portions to attach to different adjustable-length portions can further reduce the inventory costs for a medical provider while simultaneously allowing the medical provider to more easily perform different types of surgeries.

To aid in ensuring the fixed-length portions and the adjustable-length portions are connected in the correct orientation, the fixed-length portions and adjustable-length portions can be designed to include complementary keys and keyways at the location where the respective portions are joined together. For example, in one embodiment, the adjustable-length portion 150 described above (see FIG. 1A) could have a keyway designed to receive a complementary key on the fixed-length portion 110 to ensure that the fixed-length portion 110 is in the proper orientation to enable the intramedullary nail 100 (FIG. 1A) to be inserted into the femur 50 through the greater trochanter 56 of the femur 50. In one embodiment, the adjustable-length portion 150 can include two or more unique keyways designed to receive particular keys on different fixed-length portions. For example, the adjustable-length portion 150 can include a first keyway for a unique key on the fixed-length portion 110 (see FIG. 1A), a second keyway for a unique key on the fixed-length portion 210 (see FIG. 2), a third keyway for a unique key on the fixed-length portion 310 (see FIG. 3A), etc. Using a keyed connection for connecting the fixed-length portion and the adjustable-length portion can also ensure that the holes for the fasteners (e.g., fasteners 75) are all properly aligned for the fixed-length portion and the adjustable-length portion. Proper alignment of the holes for the fasteners enables the portions to be secured to the correct locations on the given bone with the fasteners oriented in anatomically safe trajectories.

Although the description above has focused on bones in the lower limb, such as the femur and tibia, the benefits of this disclosure can be expanded to any bone which can be modified (e.g., lengthened or shortened) with surgical nails. FIG. 6 shows another example of a bone, which can be treated with a surgical nail. FIG. 6 illustrates an intramedullary nail 600 positioned inside a humerus 90, according to another embodiment.

The humerus 90 is broken along a fracture 95 that separates the humerus 90 into an upper section 91 and a lower section 92. The intramedullary nail 600 can be used to move the lower section 92 relative to the upper section 91 for a variety of reasons, such as to lengthen, shorten, or assist in removing a bone defect that could be present at the fracture 95 or another location.

The intramedullary nail 600 includes a fixed-length portion 610 (also referred to as a first portion) and an adjustable-length portion 650 (also referred to as a second portion). Each portion 610, 650 is secured to the humerus 90 with fasteners 75. The fixed-length portion 610 includes holes 613 to secure the fixed-length portion 610 to the upper section 91 of the humerus 90.

The adjustable-length portion 650 includes a base portion 651 and an extendable portion 652. The extendable portion 652 can move relative to the base portion 651, for example using a telescopic arrangement and the actuator 155 described above (see FIG. 1A). The actuator 155 and the connection of the actuator 155 to the extendable portion 652 are the same as shown in FIG. 1A for the connection between the actuator 155 and the extendable portion 152 and are omitted in FIG. 6 for ease of illustration. The extendable portion 652 can extend or retract relative to the base portion 651. The extendable portion 652 includes holes 653. The fasteners 75 extend through the holes 653 to secure the extendable portion 652 to the lower section 92 of the humerus 90.

Similar to the intramedullary nail 100 described above, the intramedullary nail 600 including the adjustable-length portion 650 can be modified to use different fixed-length portions, such as fixed-length portions having a different screw configuration, fixed-length portions configured for a different insertion point on the humerus, or fixed-length portions having a different length. The fixed-length portions for the humerus are relatively inexpensive like the fixed-length portions for the femur and tibia described above. Thus, with one adjustable-length portion 650, a medical provider can obtain a large number of fixed-length portions for the humerus and be prepared to perform a wide array of surgeries (e.g., lengthening or shortening surgeries) on the humerus for a relatively low cost of inventory. When the medical provider is prepared to perform a wide variety of different types of surgery on the humerus, the likelihood of a patient having their procedure delayed (e.g., waiting for the intramedullary nail to arrive from the manufacturer) can be reduced in a similar fashion as described above for the femur and tibia. In some embodiments, the adjustable-length portion 150 described above for use on the femur and tibia can also be used for a bone, such as the humerus, which would further reduce inventory costs for a medical service provider compared to a situation where different adjustable-length portions are needed for the femur and humerus.

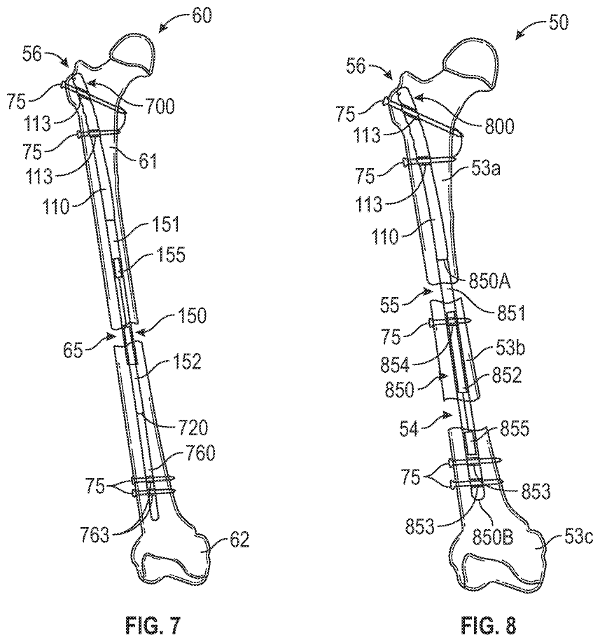

FIG. 7 illustrates an intramedullary nail 700 positioned inside the femur 60, according to another embodiment. The femur 60 is the same longer femur 60 (i.e., longer than the femur 50 shown in FIGS. 1A, 2, 3A, and 3B) described above in reference to FIG. 4. The intramedullary nail 700 includes an additional attachment to the adjustable-length portion 150 when compared to the intramedullary nails described above. This additional attachment is a fixed-length portion 760, which attaches to the extendable portion 152 of the adjustable-length portion 150. With this additional attachment, the intramedullary nail 700 illustrates another way in which the modular system can be expanded to allow for more surgeries to be performed from a limited inventory of adjustable-length portions (i.e., the expensive portions).

The intramedullary nail 400 described above in reference to FIG. 4 showed one way in which the modular system described herein can be used to form an intramedullary femoral nail for a longer femur, such as the femur 60. The intramedullary nail 400 used a longer fixed-length portion 410 for the longer femur 60 instead of the shorter fixed-length portion 110 that was used for the shorter femur 50 as part of the intramedullary nail 100 shown in FIG. 1A.

The intramedullary nail 700 includes the same fixed-length portion 110 as the intramedullary nail 100 (see FIG. 1A) described above. For the intramedullary nail 700, the fixed-length portion 110 attaches to the base portion 151 of the adjustable-length portion 150 in the same way as described above in reference to the intramedullary nail 100. Additionally, the intramedullary 700 includes the second fixed-length portion 760, which attaches to the extendable portion 152 of the adjustable-length portion 150. The second fixed-length portion 760 can attach to the extendable portion 152 using a connection 720. The connection 720 can be a similar connection (e.g., a set screw or corresponding male/female threaded parts, etc.) as described above for attaching the different fixed-length portions (e.g., fixed-length portion 110) to the base portion 151 of the adjustable-length portion 150. Alternatively, many other methods of connection could be used.

The second fixed-length portion 760 includes a set of holes 763 through which fasteners 75 can be inserted to connect the second fixed-length portion 760 with the lower section 62 of the femur 60. The holes 153 of the adjustable-length portion 150 are omitted for ease of illustration.

The second fixed-length portion 760 illustrates another way in which inventory costs can be lowered. The second fixed-length portion 760 is straight. Therefore, the second fixed-length portion 760 can be less costly than other fixed-length portions that are not straight, such as the fixed-length portion 110. Although the fixed-length portions described above in FIGS. 1A-6 can substantially reduce inventory costs, it can still be burdensome to maintain the inventory to have different lengths for each type of fixed-length portion that connects to the base portion (e.g., base portion 151) of the adjustable-length portion (e.g., adjustable-length portion 150). For example, without a component, such as the second fixed-length portion 760 that connects to the extendable portion 152 of the adjustable-length portion 150, a fully prepared inventory may need a plurality of different lengths for each of the fixed-length portions 110, 210, 310, 370, 510, and 610 described above. On the other hand, the second fixed-length portion 760 can be used on all of the intramedullary nails that used the fixed-length portions 110, 210, 310, 370, 510, and 610 described above, so it is easy to see how wide application of the second fixed-length portion 760 can further reduce inventory costs for a medical service provider.

In some embodiments the second fixed-length portion 760 is cylindrical and straight. In other embodiments the second fixed-length portion 760 may be curved, or may have an anatomic bow, or may have another complex shape.

In some embodiments, a fixed-length portion (e.g., second fixed-length portion 760) connected to the extendable end of the intramedullary nail may be the only fixed-length portion included in an intramedullary nail. For example, manufacturers presently fabricate intramedullary nails as integral units without a removable fixed-length portion, such as the fixed-length portion 110 of the intramedullary nail 100 shown in FIG. 1A. For these intramedullary nails manufactured as integral units, a fixed-length portion (e.g., second fixed-length portion 760) connected to the extendable end of the intramedullary nail still provides a way to reduce inventory costs because a medical service provider will not have to keep all of the different lengths of each different type of integral-unit intramedullary nail in stock.

Furthermore, although the second fixed-length portion 760 is shown being connected to the bottom of the extendable portion 152 of the adjustable-length portion 150 at the connection 720, in other embodiments, a fixed-length portion similar to the second fixed-length portion 760 could also be connected to the base portion 151 at the top of the adjustable-length portion 150 instead of being connected to the bottom of the extendable portion 152. For example, in one embodiment, a fixed-length portion similar to the second fixed-length portion 760 could be connected between the fixed-length portion 110 and the adjustable-length portion 150 to form an intramedullary nail of substantially the same dimensions as the intramedullary nail 700 shown in FIG. 7. This similar fixed-length portion could include threaded connections or be configured to receive a set screw at both ends to enable the connection to the adjustable-length portion 150 and to the other fixed-length portion 110 (e.g., fixed-length portion 110). Alternatively, many other methods of connection could be used. When this other fixed-length portion is installed between the fixed-length portion 110 and the adjustable-length portion 150, the cross-sectional area of the fixed-length portion could be configured to match or substantially match the cross-sectional areas of the fixed-length portion 110 and the base portion 151, so that the outer surface of the intramedullary nail is substantially the same across these different portions. For example, if the fixed-length portion 110, the base portion 151, and this other fixed-length portion are cylindrical in shape, then each of these portions can have the same diameter in some embodiments. That said, it is not required for different portions to have matching or substantially matching cross-sectional areas. For example, there may be situations where a different size, different shape, or different size and shape of a cross-sectional area of an attachment may be beneficial. For example, an attachment being installed at an anatomical location with less available space may make it beneficial to have the attachment have a smaller cross-sectional area than other portions of the surgical device.

FIG. 8 illustrates an intramedullary nail 800 positioned inside the femur 50, according to another embodiment. The intramedullary nail 800 is a bone transport nail that can be used to assist the user to reconstruct a bony defect. For example, the user may have a defect relating to a section of bone removed due to trauma, tumor, or an infection. In some embodiments, the intramedullary nail 800 can be used to reconstruct the bony defect without changing the overall length of the bone.

The femur 50 includes a first fracture 54 and a second fracture 55. The first fracture 54 is at the location where the defect occurred, and a section of bone (e.g., bone damaged by trauma, a tumor, or infection) has been removed at the location of the first fracture 54. The second fracture 55 is at a location where an osteotomy has been performed to enable the bone transport process to be performed. The first and second fractures 54, 55 have separated the femur into a first section 53a, a second section 53b, and a third section 53c. The first section 53a is located at the top of the femur 50. The third section 53c is located at the bottom of the femur 50. The second section 53b is located between the first section 53a and the third section 53c.

The intramedullary nail 800 includes the fixed-length portion 110 described above, for example in reference to the intramedullary nail 100 of FIG. 1A. For the intramedullary nail 800, the fixed-length portion 110 is referred to as the first portion 110. As discussed above, the first portion 110 includes no moving parts.

The intramedullary nail 800 further includes a second portion 850. The second portion 850 extends along a length from a first end 850A to a second end 850B. The second portion 850 is connected to the first portion 110 at the first end 850A of the second portion 850 using a similar connection (e.g., a set screw or corresponding male/female threaded parts, etc.) as described above for attaching the fixed-length portions (e.g., fixed-length portion 110) to the base portion 151 of the adjustable-length portion 150 (see FIGS. 1A-1D).

The second portion 850 is used to move the second section 53b of bone away from the first section 53a and towards the third section 53c. This movement allows new bone to be generated at the second fracture 55 until the newly generated bone (bony regenerate from distraction osteogenesis) fills in the fracture 55, while at the same time shortening the defect at fracture 54 until second section 53b and third section 53c make contact to begin to heal at the first fracture 54. The above results in a healed bone that is similar in size and strength to the bone before the defect (e.g., from trauma, a tumor, or infection, etc.) occurred.

The second portion 850 includes an outer tube 851 (first part) and an inner shell 852 (second part). The second portion further includes an actuator 855. The actuator 855 can move the inner shell 852 within the outer tube 851. For example, the actuator 855 can move the inner shell 852 towards the third section 53c of the femur 50. In some embodiments, the actuator 855 is configured to operate in both directions. In such embodiments, the actuator 855 is configured to move the inner shell 852 towards the third section 53c of the femur 50 or to move the inner shell 852 towards the first section 53a of the femur 50. Examples of suitable actuators that can be used as the actuator 855 include, but are not limited to, a magnetic actuator, hydraulic actuator, heat sensitive actuator, cold sensitive actuator, pressure sensitive actuator, motion sensitive actuator, or an electric motor. Many of the actuators that can be used as the actuator 855 can be remotely activated from outside of the patient's body.

The first portion 110 of the intramedullary nail 800 is secured to the first section 53a of femur 50 using fasteners 75 extending through holes 113 in the first portion 110. The second portion 850 is secured to the second section 53b and to the third section 53c of the femur 50. The outer tube 851 is secured to the third section 53c of femur 50 using fasteners 75 extending through holes 853 in the outer tube 851. The inner shell 852 is secured to the second section 53b of femur 50 using one or more fasteners 75 extending through one or more holes 854 in the inner shell 852. The outer tube 851 can include a slot (not shown) extending along a length of the second portion 850 (i.e., in the direction from the first end 850A to the second end 850B) to allow the inner shell 852 and the fastener 75 extending through the hole 854 to move relative to the outer tube 851. This slot allowing movement of the inner shell 852 and fastener 75 relative to the outer tube 851 enables the actuator 855 to cause the second section 53b of the femur 50 to move away from the first section 53a of the femur 50 and towards the third section 53c of femur 50.

The intramedullary nail 800 is a bone transport nail but uses the same fixed-length portion 110 as the bone lengthening/shortening intramedullary nail 100 described above in reference to FIG. 1A. Furthermore, the intramedullary nail 800 can be modified in a similar way to all of the variations of the intramedullary nail 100 described above in FIGS. 2-6. For example, the first portion 110 of the intramedullary nail 800 could be replaced with (1) the portion 210 having a different fastener configuration (see FIG. 2A), (2) the portion 310 having a different shape (see FIG. 3A) allowing for a different insertion point on the femur 50, (3) the portion 370 (see FIG. 3B) allowing for a retrograde insertion instead of an antegrade insertion, (4) the portion 410 having a larger size (see FIG. 4), or (5) the portion 510 (see FIG. 5) or the portion 610 (see FIG. 6) allowing for insertion into different bones, such as the tibia or humerus.

Furthermore, in some embodiments, the second portion 850 can also be configured to make a connection to another attachment at the second end 850B of the second portion 850 similar to how the intramedullary nail 700 included a connection to the second fixed-length portion 760. For example, the second portion 850 can be connected to a third portion (not shown) to extend the length of the intramedullary nail 800 similar to how the second fixed-length portion 760 extended the length of the intramedullary nail 700 (see FIG. 7) relative to the length of the intramedullary nail 100 (see FIG. 1A).

Thus, the modular system described herein can be applied to bone lengthening/shortening nails as well as to bone transport nails. Therefore, the inventory cost savings discussed above for using a modular system for bone lengthening/shortening nails also can be applied to achieve similar inventory cost savings when a modular system is applied to bone transport nails. Furthermore, in some applications the same components (e.g., the fixed-length portion 110) can be used as part of either a bone lengthening/shortening intramedullary nail (e.g., intramedullary nail 100 of FIG. 1A) or as part of a bone transport intramedullary nail (e.g., intramedullary nail 800 of FIG. 8). The ability to use same part (e.g., fixed-portion 110) as part of either a bone lengthening/shortening nail or as part of a bone transport nail only further serves to reduce inventory costs for a medical services provider and reduces the likelihood that a patient will have to wait for a custom intramedullary nail to arrive from a manufacturer.

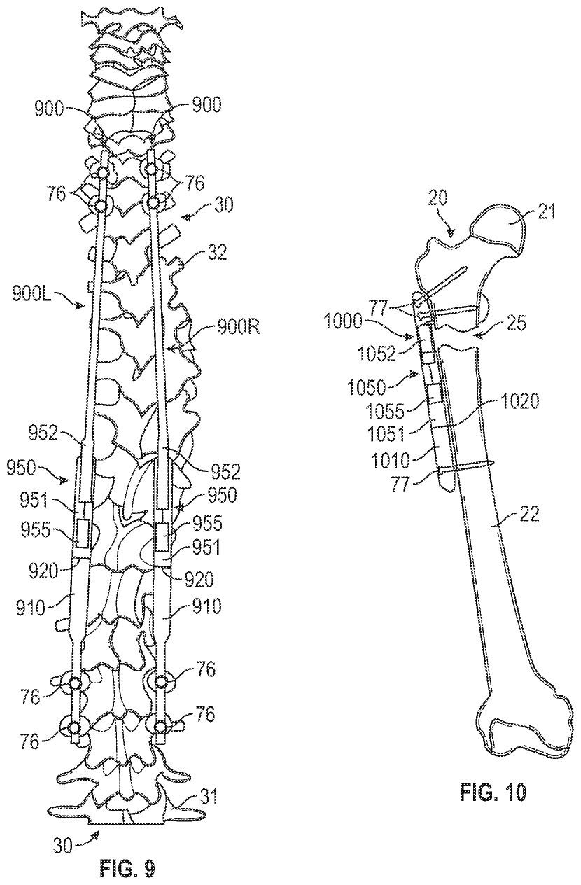

Although the description above has mainly focused on modular systems for a variety of intramedullary devices for use inside a patient's bone, the benefits of this disclosure can also apply to extramedullary devices for use outside of a patient's bone. Two non-limiting examples are provided below in reference to FIGS. 9 and 10.

FIG. 9 illustrates a pair of spinal rods 900, according to another embodiment. The pair of spinal rods 900 include a left spinal rod 900L and a right spinal rod 900R. The spinal rods are fixed to a spine 30.

Each spinal rod 900 includes a fixed-length portion 910 and an adjustable-length portion 950. The portions 910, 950 in each spinal rod 900L, 900R can be attached to each other at a connection 920. Fasteners 76 (e.g., pedicle screws) can be used to attach each fixed-length portion 910 to a lower portion 31 of the spine 30. Fasteners 76 can also be used to attach each adjustable-length portion 950 to an upper portion 32 of the spine 30.

Each adjustable-length portion 950 can include a base portion 951 and an extendable portion 952. The extendable portion 952 can move relative to the base portion 951, for example using a telescopic arrangement. For example, the extendable portion 952 can extend or retract relative to the base portion 951.

Each base portion 951 includes an actuator 955. The actuator 955 can be activated to move the extendable portion 952 relative to the base portion 951. After the spinal rod 900 is secured to the spine 30 as shown, the movement of the extendable portion 952 initiated by the actuator 955 can be used to achieve the desired outcome (e.g., straightening of the spine). The actuator 955 can be any type of actuator suitable for creating movement of the extendable portion 952 relative to the base portion 951. Examples of suitable actuators that can be used as the actuator 955 include, but are not limited to, a magnetic actuator, hydraulic actuator, heat sensitive actuator, cold sensitive actuator, pressure sensitive actuator, motion sensitive actuator, or an electric motor. Many of the actuators that can be used as the actuator 955 can be remotely activated from outside of the patient's body.

The fixed-length portion 910 is removably attached to the adjustable-length portion 950, for example in a similar manner described above in reference to attaching the fixed-length portion 110 to the adjustable-length portion 150 for the intramedullary nail 100 (see FIGS. 1A-1C). A modular system can be applied to the spinal rods 900 in a similar way the modular systems described above were applied to the various intramedullary nails. For example, a modular system for the spinal rods 900 can include but is not limited to (1) fixed-length portions having different lengths and diameters, (2) fixed-length portions having different shapes (e.g., different shapes may be better depending on the curve of a particular patient's spine), and (3) fixed-length portions configured for different fastener configurations. A modular system for the spinal rods can also include attachments for connecting to the extendable portion 952 that are similar to the second fixed-length portion 760, which attached to the extendable portion 152 of the intramedullary nail 700 described above in reference to FIG. 7.

FIG. 10 illustrates a surgical plate 1000, according to another embodiment. The surgical plate is externally fixed to a femur 20. The femur includes a fracture 25 separating the femur 20 into an upper portion 21 and a lower portion 22.

The surgical plate 1000 includes a fixed-length portion 1010 and an adjustable-length portion 1050. The portions 1010, 1050 are attached to each other at a connection 1020. Fasteners 77 (e.g., screws) can be used to attach the fixed-length portion 1010 and the adjustable-length portion 1050 to different portions of the femur 20.

The adjustable-length portion 1050 can include a base portion 1051 and an extendable portion 1052. The extendable portion 1052 can move relative to the base portion 1051. For example, the extendable portion 1052 can extend or retract relative to the base portion 1051.

The base portion 1051 includes an actuator 1055. The actuator 1055 can be activated to move the extendable portion 1052 relative to the base portion 1051. After the surgical plate 1000 is secured to the femur 20 as shown, the movement of the extendable portion 1052 initiated by the actuator 1055 can be used to achieve the desired outcome (e.g., bone lengthening or bone shortening). The actuator 1055 can be any type of actuator suitable for creating movement of the extendable portion 1052 relative to the base portion 1051. Examples of suitable actuators that can be used as the actuator 1055 include, but are not limited to, a magnetic actuator, hydraulic actuator, heat sensitive actuator, cold sensitive actuator, pressure sensitive actuator, motion sensitive actuator, or an electric motor. Many of the actuators that can be used as the actuator 1055 can be remotely activated from outside of the patient's body.

The fixed-length portion 1010 is removably attached to the adjustable-length portion 1050 at the connection 1020, for example in a similar manner described above in reference to attaching the fixed-length portion 110 to the adjustable-length portion 150 for the intramedullary nail 100 (see FIGS. 1A-1C). A modular system can be applied to the surgical plate 1000 in a similar way the modular systems described above were applied to the various intramedullary nails. For example, a modular system for the surgical plate 1000 can include but is not limited to (1) fixed-length portions having different lengths and thicknesses, (2) fixed-length portions having different shapes (e.g., different shapes may be better depending on the bone being treated as well as the location on the bone being treated), and (3) fixed-length portions configured for different fastener configurations.

A modular system for the surgical plate 1000 can also include attachments (e.g., fixed-length attachments) for connecting to the extendable portion 1052 that are similar to the second fixed-length portion 760, which attached to the extendable portion 152 of the intramedullary nail 700 described above in reference to FIG. 7. Thus, the modular system for the surgical plate 1000 can include attachments at both ends of the adjustable-length portion 1050, such as the fixed-length portion 1010 shown in FIG. 10 and another attachment (not shown) at the top of the surgical plate 1000.

In another embodiment, a modular system for surgical plates that can function as bone transport plates is also envisioned by this disclosure. These bone transport plates can function in a similar fashion to that described above for bone transport nail 800 of FIG. 8. For example, an exemplary bone transport surgical plate for this modular system could include the fixed-length portion 1010 and a second portion. The second portion could include an actuator and parts that are movable relative to each other by the actuator similar to how the inner shell 852 was movable relative to the outer tube 851 for the bone transport intramedullary nail 800 described above. This modular system of bone transport plates also envisions bone transport plates with attachments at both ends of the second portion of the bone transport plate similar to how the intramedullary nail 700 included the fixed-length portion 110 and the second fixed-length portion 760 at either end of the intramedullary nail 700. Furthermore, there could be two or more fixed-length attachments at both ends of the second portion of the bone transport plate.

Furthermore, in some embodiments, all of the intramedullary nails described above along with all of the combinations of attachments described above can instead be used as extramedullary surgical nails that are secured to the outside of a patient's bone. In some of these embodiments, the size, shape, and/or fastener configuration of these extramedullary surgical devices may change relative to the corresponding intramedullary embodiments described above to facilitate installation of the surgical devices outside of a patient's bone instead of inside of a patient's bone.

As mentioned above in reference to FIG. 7, the intramedullary nail 700 can include two or more fixed-length attachments at either end of adjustable-length portion 150. Furthermore, each surgical device disclosed herein can be modified to include two or more fixed-length attachments at either end of the portion that includes the actuator. Having the modular systems described herein configured to accommodate two or more attachments at either end of the portion including the actuator (e.g., adjustable-length portion 150) only further serves to increase the flexibility of the modular system, reduce inventory costs, and reduce the likelihood of a patient waiting for a custom surgical device arriving from the manufacturer.

Additionally, each portion of a surgical device without an actuator (e.g., fixed-length portion 110 from FIG. 1A) described in this disclosure can be formed of a single unitary portion or formed of two or more sub-portions. In embodiments in which two or more sub-portions are used to form a portion (e.g., fixed-length portion 110) of a surgical device, one or more of the sub-portions can still be attached to the other portion (e.g., adjustable-length portion 150 from FIG. 1A) without any of the other sub-portions and still form a functioning surgical device. For example, in one embodiment the lower 20% of the length of the fixed-length portion of FIG. 1A could be a separate removable sub-portion. The remaining portion of the fixed-length portion 110 (i.e., the other 80%) could then be attached to the adjustable-length portion 150 for use on a femur that is shorter than the femur 50 shown in FIG. 1A. This use of sub-portions is another way in which the modular systems described herein can enhance the ability to customize the dimensions and shape of portions (e.g., fixed-length portions) of the surgical devices described herein to more appropriately match a patient's anatomy and/or injury.

Many of the movable portions described herein have generally been described as moving along a straight line relative to another portion. For example, the extendable portion 152 (see e.g., FIG. 1A) can move along a straight line relative to the base portion 151 of the adjustable-length portion 150. Similarly, the movable portions in surgical devices in FIGS. 7-10 can also move along a straight line relative other portions in those devices. In other embodiments, the surgical devices described above can be modified to have movable portions that can include a curve, bow, or other non-straight feature allowing for movement along a path other than a straight path. Having a movable portion that can move along a path other than a straight path can be useful, for example in a portion of a bone that curves as well as for other reasons.

While the foregoing is directed to embodiments of the present disclosure, other and further embodiments of the disclosure may be devised without departing from the basic scope thereof, and the scope thereof is determined by the claims that follow.

1. A modular system for a surgical device, the modular system comprising:
an adjustable-length portion having a first end and a second end, the first end spaced apart from the second end along a length of the adjustable-length portion, and the adjustable-length portion including an actuator configured to be activated to change the length of the adjustable-length portion, wherein the adjustable-length portion is configured to be secured to one or more bones; and
a first fixed-length portion configured to attach to the first end of the adjustable-length portion to form a first surgical device, wherein the first fixed-length portion is configured to attach to the first end of the adjustable-length portion using a first fastener aligned in a same direction as the length of the adjustable-length portion.

2. The modular system of claim 1, wherein the first surgical device is an intramedullary nail configured to lengthen a bone.

3. The modular system of claim 1, wherein the first surgical device is an intramedullary nail configured to shorten a bone.

4. The modular system of claim 1, further comprising a second fixed-length portion, wherein
the first fixed-length portion has a first end and a second end,
the second end of the first fixed-length portion is connected to the adjustable-length portion, and
the second fixed-length portion is configured to attach to the first end of the first fixed-length portion.

5. The modular system of claim 1, wherein the actuator is configured to move the second end of the adjustable-length portion to change the length of the adjustable-length portion.

6. The modular system of claim 5, further comprising a second fixed-length portion configured to attach to the second end of the adjustable-length portion.

7. The modular system of claim 6, further comprising a third fixed-length portion wherein
the first fixed-length portion has a first end and a second end,
the second end of the first fixed-length portion is connected to the adjustable-length portion, and
the third fixed-length portion is configured to attach to the first end of the first fixed-length portion.

8. The modular system of claim 1, further comprising a second fixed-length portion configured to attach to the first end of the adjustable-length portion to form a second surgical device, wherein the first fixed-length portion has a different size, a different shape, or a different fastener configuration than the second fixed-length portion.

9. The modular system of claim 8, wherein
the first fixed-length portion includes one or more holes, each hole configured to receive a corresponding fastener, the one or more holes and corresponding one or more fasteners configured to attach the first fixed-length portion to one or more first locations on a first bone; and
the second fixed-length portion includes one or more holes, each hole configured to receive a corresponding fastener, the one or more holes and corresponding one or more fasteners configured to attach the second fixed-length portion to one or more second locations on the first bone.

10. The modular system of claim 8, wherein the first fixed-length portion has a different shape than the second fixed-length portion.

11. The modular system of claim 10, wherein
the first surgical device is configured to be substantially fully inserted into a first bone through a first location on the first bone,
the second surgical device is configured to be substantially fully inserted into the first bone through a second location on the first bone,
the first surgical device is not configured to be substantially fully inserted into the first bone at the second location, and
the second surgical device is not configured to be substantially fully inserted into the first bone at the first location.

12. The modular system of claim 11, wherein the first location is at a first end of the first bone and the second location is at a second end of the first bone.

13. The modular system of claim 8, wherein the first fixed-length portion has a different size than the second fixed-length portion.

14. The modular system of claim 8, wherein the first fixed-length portion has a different size and shape than the second fixed-length portion.

15. The modular system of claim 1, wherein the first surgical device is an extramedullary spinal rod.

16. The modular system of claim 1, wherein the first surgical device is an extramedullary plate or rod.

17. A modular system for a surgical device, the modular system comprising:
an adjustable-length portion having a first end and a second end, the first end spaced apart from the second end along a length of the adjustable-length portion, the adjustable-length portion configured to be secured to one or more bones, and the adjustable-length portion including an actuator configured to be activated to change the length of the adjustable-length portion by moving the second end; and
a first fixed-length portion configured to attach to the second end of the adjustable-length portion to form a first surgical device.

18. The modular system of claim 17, wherein the first surgical device is an intramedullary nail configured to lengthen a bone.

19. The modular system of claim 17, wherein the first surgical device is an intramedullary nail configured to shorten a bone.

20. The modular system of claim 17, further comprising a second fixed-length portion, wherein
the first fixed-length portion has a first end and a second end,
the first end of the first fixed-length portion is connected to the adjustable-length portion, and
the second fixed-length portion is configured to attach to the second end of the first fixed-length portion.

21. The modular system of claim 17, further comprising a second fixed-length portion configured to attach to the second end of the adjustable-length portion to form a second surgical device, wherein the first fixed-length portion has a different size, a different shape, or a different fastener configuration than the second fixed-length portion.

22. The modular system of claim 21, wherein a third surgical device is formed through attachment of a third fixed-length portion to the first end of the adjustable-length portion and attachment of the first fixed-length portion to the second end of the adjustable-length portion, wherein the third fixed-length portion is configured to attach to the first end of the adjustable-length portion using a first fastener aligned in a same direction as the length of the adjustable-length portion.

23. The modular system of claim 22, wherein a fourth surgical device is formed through attachment of the third fixed-length portion to the first end of the adjustable-length portion and attachment of the second fixed-length portion to the second end of the adjustable-length portion.

24. A modular system for a surgical device, the modular system comprising:
a first portion; and
a second portion having a first end and a second end, the first end spaced apart from the second end along a length of the second portion, the second portion including a first part, a second part, and an actuator configured to move the first part relative to the second part, wherein
the second portion is configured to be secured to one or more bones,
the first portion is configured to attach to the first end of the second portion to form a first surgical device, and
the first portion is configured to attach to the first end of the second portion using a first fastener aligned in a same direction as the length of the second portion.

25. The modular system of claim 24, wherein the second part is an outer tube and the first part is an inner shell positioned at least partially inside the outer tube.

26. The modular system of claim 24, wherein the first surgical device is an intramedullary nail.

27. The modular system of claim 26, wherein the first surgical device is a bone transport nail.

28. The modular system of claim 24, further comprising a third portion, wherein
the first portion has a first end and a second end,
the second end of the first portion is connected to the second portion, and
the third portion is configured to attach to the first end of the first portion.

29. The modular system of claim 24, further comprising a third portion configured to attach to the second end of the second portion.

30. The modular system of claim 29, further comprising a fourth portion, wherein
the first portion has a first end and a second end,
the second end of the first portion is connected to the second portion, and
the fourth portion is configured to attach to the first end of the first portion.

31. The modular system of claim 24, further comprising a third portion, wherein
the third portion is configured to attach to the first end of the second portion to form a second surgical device, and
the first portion has a different size, a different shape, or a different fastener configuration than the third portion.

32. The modular system of claim 31, wherein the first portion has a different shape than the third portion.

33. The modular system of claim 31, wherein the first portion has a different size than the third portion.

34. The modular system of claim 31, wherein the first portion has a different size and shape than the third portion.

35. The modular system of claim 31, further comprising a fourth portion configured to attach to the second end of the second portion.

36. The modular system of claim 35, wherein a third surgical device is formed by attachment of the first portion to the first end of the second portion and attachment of the fourth portion to the second end of the second portion.

37. The modular system of claim 36, wherein a fourth surgical device is formed by attachment of the third portion to the first end of the second portion and attachment of the fourth portion to the second end of the second portion.

38. The modular system of claim 24, wherein the first surgical device is an extramedullary surgical device.

39. The modular system of claim 38, wherein the first surgical device is a bone transport nail.
